(19) United States
(12) Patent Application Publication
(10) Pub. No.: US 2026/0074038 A1
(43) Pub. Date: Mar. 12, 2026

(54) Artificial Intelligence-Driven Digital Health Platform

(71) Applicant: knowRX, Inc., Austin, TX (US)

(72) Inventor: David Franklin, Austin, TX (US)

(21) Appl. No.: 19/326,539

(22) Filed: Sep. 11, 2025

Related U.S. Application Data
(60) Provisional application No. 63/693,556, filed on Sep. 11, 2024.

(51) Int. Cl. G16H 10/60; G06Q 20/20

(52) U.S. Cl. CPC G16H 10/60; G06Q 20/20

(57) ABSTRACT

A digital health platform may be configured to manage health data. The platform may receive, from a mobile application associated with a user, first health data including at least one of a prescription indicator or a user-reported health metric. Second health data including at least one physiological measurement may be received from at least one wearable device associated with the user. Third health data associated with the user may be received from at least one external health record data source. The first health data, the second health data, and the third health data may be aggregated into a Consumer Health Record (CHR). A bi-directional exchange of at least a portion of the CHR with an external healthcare system may be facilitated via a secure data exchange system based on governance rules set by the user.

### CROSS-REFERENCE TO RELATED APPLICATION(S)

This application claims the benefit of priority from U.S. Provisional Application Ser. No. 63/693,556, filed Sep. 11, 2024, the entire disclosure of which is herein incorporated by reference.

### FIELD

The present disclosure relates data processing platforms, and more particularly to healthcare data management.

### BRIEF DESCRIPTION OF THE DRAWINGS

This disclosure is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity.

FIG. 1 is a block diagram of an example of a computing environment for providing artificial-intelligence and/or machine learning (AI/ML)-driven digital health management.

FIG. 2 is a block diagram of an example internal configuration of a computing device.

FIG. 3 is a flow diagram illustrating an example of a technique associated with providing AI/ML-driven digital health management.

### DETAILED DESCRIPTION

Modern healthcare data management systems face technical challenges in aggregating and utilizing patient health information from disparate sources. Existing systems, such as an Electronic Medical Record (EMR) system or an Electronic Health Record (EHR) system, are often institutionally controlled and store clinical data in siloed repositories. This architectural limitation creates data fragmentation, where health information captured during clinical visits is disconnected from the vast amount of real-world data generated by a user's daily activities. The lack of integration with data from sources like personal wearable devices, mobile health applications, and patient-reported outcomes results in an incomplete and episodic view of a user's health status.

The technical problem is further compounded by the static nature of these conventional systems. They are not configured to process data streams continually, periodically, or in response to a trigger event, which may be beneficial for proactive health management and personalized care. For instance, data from wearable devices, which may include physiological measurements, or user-reported health metrics from mobile applications, are not seamlessly incorporated into a patient's primary health record. This technological gap hampers the ability of healthcare providers to make fully informed decisions based on a holistic understanding of a patient's health journey, limiting care to reactive interventions rather than proactive, predictive health management.

Consequently, there are limitations in tracking medication adherence, predicting adverse health events, and generating personalized care plans. The inability of current computer-implemented systems to aggregate, normalize, and analyze data from multiple, heterogeneous sources-including clinical records, pharmacy systems, wearable devices, and genomic data-prevents the application of advanced analytics, such as artificial intelligence (AI) and/or machine learning (ML) (AI/ML) algorithms, at their full potential. This results in a system that cannot effectively provide dynamic, data-driven insights to users, healthcare providers, or industry partners, nor can it support a secure, user-controlled framework for data exchange and governance.

Implementations of this disclosure address problems such as these by providing a digital health platform configured to manage a comprehensive Consumer Health Record (CHR), which integrates data from diverse sources to facilitate AI-driven personalized care and secure, user-governed data exchange. The disclosed subject matter may be configured for receiving, from a mobile application associated with a user, first health data including at least one of a prescription indicator or a user-reported health metric. The digital health platform may also be configured for receiving, from at least one wearable device associated with the user, second health data including at least one physiological measurement. Furthermore, the digital health platform may be configured for receiving, from at least one external health record data source, third health data associated with the user.

As used herein, the term “Consumer Health Record (CHR)” may refer to a comprehensive, user-centric digital compilation of health-related information aggregated from multiple sources. For example, a CHR may include data from clinical visits, pharmacy records, self-reported metrics, wearable devices, and genomic tests. In some implementations, the CHR is a dynamic record that is continually, periodically, or in response to a trigger event updated with real-time information, providing a holistic view of the user's health. The digital health platform may be configured for aggregating the first health data, the second health data, and the third health data into the CHR.

The disclosed subject matter facilitates a secure, bi-directional exchange of health information. A secure data exchange system may be configured for facilitating a bi-directional exchange of at least a portion of the CHR with an external healthcare system based on governance rules set by the user. As used herein, the term “governance rules” may refer to a set of user-defined permissions and constraints that dictate how, with whom, and for what duration their health data may be shared. For example, a user may set a governance rule to provide a read-only view of their CHR to a specific healthcare provider for a limited time. In some implementations, the governance rules may include an expiration date for data access. The digital health platform may determine the governance rules based on accessing a dynamic consent management system that facilitates modification, by the user, of data sharing permissions. Facilitating the bi-directional exchange may be initiated via at least one of a quick response (QR) code or a web portal.

The external health record data source may include at least one of an EMR system or an EHR system. The digital health platform may also integrate with other external healthcare systems, such as a pharmacy Point-of-Sale (POS) system. In implementations where the external healthcare system includes a pharmacy POS system, the digital health platform may be configured for tracking medication adherence based on data exchanged with the pharmacy POS system. This data exchange facilitates a more complete picture of a user's medication regimen and compliance.

The present disclosure incorporates advanced analytical capabilities. The digital health platform may be configured for generating a personalized care plan based on analyzing the CHR using at least one of an AI algorithm or a machine learning (ML) algorithm. Analyzing the CHR may include predicting a potential medication side effect. Based on this analysis, the digital health platform may transmit the personalized care plan to at least one of a healthcare provider or a pharmacist. The digital health platform may also be configured for generating granular analytics based on the CHR, where the granular analytics include at least one of a health trend insight, a goal setting monitor, or a data-driven engagement tool.

To enhance security and transparency, the digital health platform may be configured for maintaining, using a blockchain component, an audit trail of transactions related to the bi-directional exchange of the at least a portion of the CHR. This creates an immutable record of data access and sharing activities. The present disclosure also provides mechanisms for user empowerment and data monetization. For example, the digital health platform may provide, via a governance rule setting, an option for the user to opt-in to a data-sharing agreement with an industry partner in exchange for compensation, wherein data shared with the industry partner is de-identified to protect user privacy.

In some implementations, the digital health platform may integrate additional data types to enrich the CHR. For example, the digital health platform may receive fourth health data including consumer genomics data and aggregate the fourth health data into the CHR. Based on this integrated data, the digital health platform may provide a personalized medicine insight based on the consumer genomics data, wherein the personalized medicine insight includes a drug interaction prediction. The digital health platform may also be configured to transmit a drug recall notification to at least one of the user or a prescribing physician based on the CHR. To further support medication adherence, the digital health platform may transmit a short message service (SMS) notification to an accountability partner designated by the user based on a medication adherence metric derived from the CHR.

In some implementations, the digital health platform may facilitate innovative healthcare delivery models. For instance, the digital health platform may facilitate medication distribution based on a crowdsourcing model. The digital health platform may also facilitate remote user monitoring by sharing the second health data with a telehealth system during a virtual consultation, thereby improving access to care and facilitating health oversight by providers.

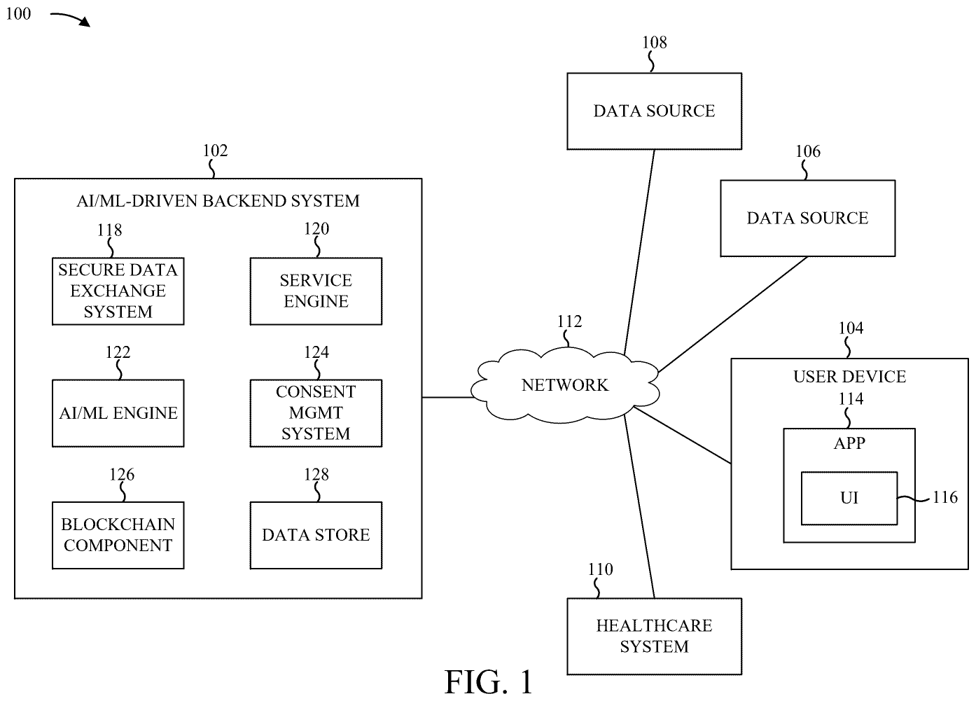

FIG. 1 is a block diagram of an example of a computing environment for providing artificial-intelligence and/or machine learning (AI/ML)-driven digital health management. The computing environment 100 includes an AI/ML-driven backend system 102, a user device 104, a data source 106, a data source 108, a healthcare system 110, and a network 112. The components of the computing environment 100 may be communicatively coupled via the network 112, facilitating data exchange and processing for managing a Consumer Health Record (CHR).

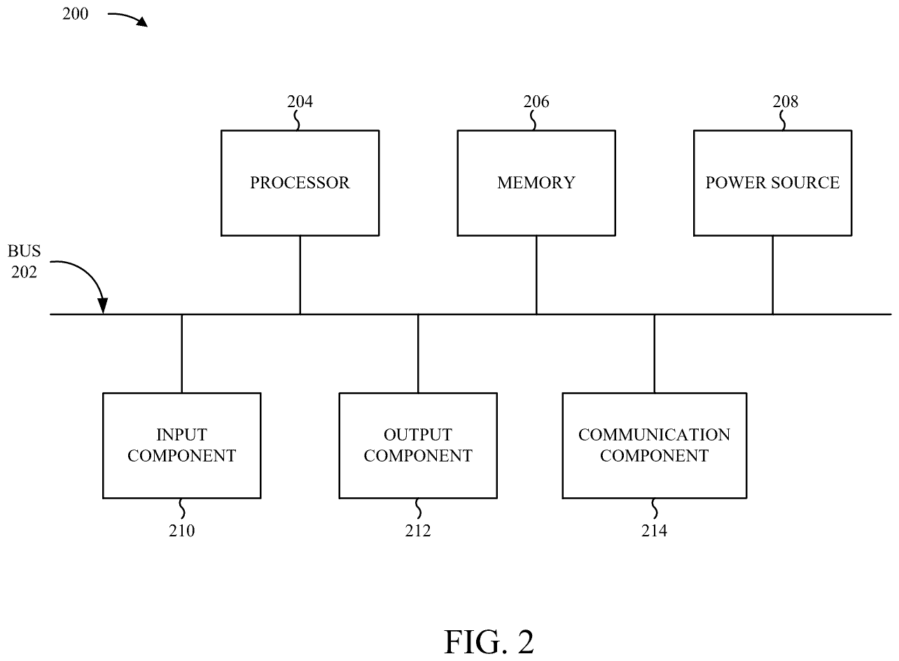

The AI/ML-driven backend system 102 (which may also be referred to as a digital health platform) may be configured to aggregate, process, and analyze health data from multiple sources to provide personalized care and manage data exchange. The AI/ML-driven backend system 102 may be implemented as one or more servers, a cloud-based computing platform, or a distributed network of computing devices. It may be configured to communicate with the user device 104, the data source 106, the data source 108, and the healthcare system 110 via the network 112 to perform the functions of the present disclosure. The AI/ML-driven backend system 102 may be implemented using one or more computing devices such as the computing device 200 described in connection with FIG. 2.

In some implementations, the AI/ML-driven backend system 102 may be configured for receiving, from a mobile application associated with a user, first health data including at least one of a prescription indicator or a user-reported health metric. It may also receive second health data from at least one wearable device, and third health data from at least one external health record data source. The AI/ML-driven backend system 102 may be configured for aggregating this data into a CHR and facilitating a bi-directional exchange of the CHR with an external healthcare system. For example, the AI/ML-driven backend system 102 may be implemented on a scalable cloud infrastructure, such as Amazon Web Services (AWS) or Microsoft Azure, utilizing microservices architecture to manage different functionalities.

The user device 104 may be a computing device associated with a user, configured to interact with the AI/ML-driven backend system 102. Examples of the user device 104 include, but are not limited to, a smartphone, a tablet computer, a laptop computer, or a desktop computer. The user device 104 may be configured to execute a mobile application that facilitates user interaction with the digital health platform, allowing for data input, visualization of health analytics, and management of data sharing permissions. The user device 104 may be, be similar to, include, or be included in the computing device 200 described in connection with FIG. 2.

The user device 104 may be configured to transmit first health data to the AI/ML-driven backend system 102 via the network 112. This data may be entered by a user through a graphical user interface. In some implementations, the user device 104 may connect to wearable devices via Bluetooth or other short-range communication protocols to collect and relay physiological measurements. For example, a user may utilize the user device 104 to scan a prescription label, report daily symptoms, or view a personalized care plan generated by the AI/ML-driven backend system 102.

The data source 106 may represent a source of real-world health data associated with the user. In some implementations, the data source 106 includes at least one wearable device, such as a smartwatch, a fitness tracker, a continuous glucose monitor, or a smart blood pressure cuff. The data source 106 may be configured to continually, periodically, or in response to a trigger event collect physiological measurements and transmit this second health data to the user device 104 or directly to the AI/ML-driven backend system 102 via the network 112.

The data source 106 provides a stream of objective, real-time data that enriches the CHR. For example, the data source 106 may be a Fitbit device that tracks heart rate, sleep patterns, and physical activity, or a Dexcom continuous glucose monitor that provides blood sugar readings. This data may be used by the AI/ML-driven backend system 102 to monitor health trends, assess medication adherence, and generate predictive insights.

The data source 108 may represent an external health record data source that stores clinical or other health-related information associated with the user. The data source 108 may include, but is not limited to, an EMR system, an EHR system, a pharmacy POS system, or a repository for consumer genomics data. As used herein, the term “Electronic Medical Record (EMR) system” may refer to a digital version of the paper charts in a clinician's office. An EMR system contains the medical and treatment history of the patients in one practice. An EMR system may be configured to track data over time, identify patients who are due for preventive screenings or checkups, and monitor how patients measure up to certain parameters such as vaccinations and blood pressure readings. An EMR system, however, is not typically designed to be shared outside the individual practice.

In contrast, as used herein, the term “Electronic Health Record (EHR) system” may refer to a digital record of health information designed to be shared among different healthcare providers. An EHR system includes data from all clinicians involved in a patient's care, including labs, specialists, and other healthcare facilities. This broader scope facilitates a more comprehensive, long-term view of a patient's health. While both EMR systems and EHR systems are sources of clinical data, EHR systems are structured for greater interoperability, which may facilitate the aggregation of data from multiple clinical settings into the Consumer Health Record (CHR). The AI/ML-driven backend system 102 may be configured to communicate with the data source 108 to receive third health data associated with the user, such as clinical visit summaries, lab results, prescription fill history, or genetic information.

In some implementations, the AI/ML-driven backend system 102 integrates with the data source 108 using standardized protocols such as Fast Healthcare Interoperability Resources (FHIR) or Health Level Seven (HL7). For example, the data source 108 may be an Epic or Cerner EHR system from which the AI/ML-driven backend system 102 pulls a user's medical history after receiving appropriate authorization. In another example, the data source 108 may be a Walgreens pharmacy POS system that provides data for tracking medication adherence.

The healthcare system 110 may be an external healthcare system that interacts with the AI/ML-driven backend system 102 to exchange health information. Examples of the healthcare system 110 include, but are not limited to, a hospital's EMR system, a telehealth platform, a pharmacist's management system, a healthcare provider's management system, or a clinical research organization's data platform. The AI/ML-driven backend system 102 may be configured to facilitate a bi-directional exchange of at least a portion of the CHR with the healthcare system 110 based on governance rules set by the user.

For example, a healthcare provider using the healthcare system 110 may be granted a read-only view of a user's CHR to inform clinical decision-making during a consultation. In another example, the AI/ML-driven backend system 102 may transmit a personalized care plan or a notification about a predicted adverse drug reaction to the healthcare system 110 for review by a physician or pharmacist. The healthcare system 110 may also be, be similar to, include, or be included in the data source 108 in some scenarios.

The network 112 may be any suitable network or combination of networks that facilitates communication between the components of the computing environment 100. The network 112 may include, for example, a local area network (LAN), a wide area network (WAN), the Internet, a cellular network, or a combination thereof. All communications between the AI/ML-driven backend system 102, the user device 104, the data source 106, the data source 108, and the healthcare system 110 may be transmitted over the network 112 using secure protocols, such as Transport Layer Security (TLS), to protect data privacy and integrity.

As shown in FIG. 1, the user device 104 includes an application 114 (shown as “app”) and a user interface (UI) 116. In some implementations, two or more of the app 114 and the UI 116 may be integrated into a single component. For example, the application 114 and the UI 116 may be part of a single software application executable on the user device 104. In some implementations, one or more of the application 114 and the UI 116 may be implemented using any number of computing devices such as the computing device 200 shown in FIG. 2. In some implementations, the application 114 may provide or otherwise include the UI 116.

The application 114 may be a mobile application or a web-based application executed on the user device 104. The application 114 provides the primary interface for the user to interact with the AI/ML-driven backend system 102. The app 114 may be configured to receive user input, display information from the CHR, present personalized care plans, and facilitate management of consent and data sharing settings. For example, the app 114 may be a native application downloaded from an application store onto a smartphone.

The UI 116 may be the graphical interface presented by the app 114 on the user device 104. The UI 116 may be configured to render screens, forms, dashboards, and notifications that facilitate user interaction. For example, the UI 116 may present a dashboard showing health trend insights, a form for logging user-reported health metrics, or a settings page for modifying governance rules for data sharing.

As shown in FIG. 1, the AI/ML-driven backend system 102 includes a secure data exchange system 118, a service engine 120, an AI/ML engine 122, a consent management system 124, a blockchain component 126, and a data store 128. In some implementations, two or more of the secure data exchange system 118, the service engine 120, the AI/ML engine 122, the consent management system 124, the blockchain component 126, and the data store 128 may be integrated into a single component. In some implementations, one or more of these components may be implemented using any number of computing devices such as the computing device 200 shown in FIG. 2. For example, these components may be distributed among a number of computing devices as a set of microservices operating in a cloud computing environment.

The secure data exchange system 118 may be configured to facilitate the bi-directional exchange of at least a portion of the CHR with an external healthcare system, such as the healthcare system 110, based on governance rules set by the user. The secure data exchange system 118 may implement secure communication protocols and application programming interfaces (APIs) to manage data flow. For example, facilitating the bi-directional exchange may be initiated via at least one of a QR code or a web portal, which the secure data exchange system 118 may generate and manage.

In some implementations, the secure data exchange system 118 is configured to operate using a set of APIs, such as those based on Representational State Transfer (REST) or FHIR standards. These APIs may facilitate authenticated and authorized data requests between the AI/ML-driven backend system 102 and an external healthcare system 110. For instance, when a user initiates a data exchange via a QR code generated for a user's CHR, the healthcare system 110 may scan the code to obtain a temporary, single-use access token. The secure data exchange system 118 may then validate this token against the governance rules stored in the data store 128 and managed by the consent management system 124 before granting access to the specified portion of the CHR.

The secure data exchange system 118 may be further configured to handle data transformation and formatting to ensure interoperability between disparate systems. For example, if the AI/ML-driven backend system 102 stores CHR data in a proprietary format, the secure data exchange system 118 may translate outgoing data into a standardized format like Health Level Seven (HL7) before transmitting it to the healthcare system 110. Conversely, when receiving data from an external source, such as an EMR system, the secure data exchange system 118 may be configured to parse the incoming HL7 or FHIR-formatted data and map it to the internal data structure of the CHR within the data store 128. This process ensures seamless integration and data consistency across the platform.

To protect data in transit, the secure data exchange system 118 may employ robust encryption protocols, such as TLS. The system may also be configured to manage data segmentation based on the governance rules. For example, a user may set a rule to share only their medication adherence data and recent physiological measurements, but not their genomic data. The secure data exchange system 118 may be configured to dynamically filter the CHR data based on these user-defined permissions, creating a tailored data package for each exchange. Each transaction facilitated by the secure data exchange system 118 may trigger a corresponding entry in the audit trail maintained by the blockchain component 126, ensuring a transparent and verifiable record of all data sharing activities.

The service engine 120 may be configured to manage the core business logic and services of the AI/ML-driven backend system 102. The service engine 120 may orchestrate data aggregation from the user device 104, the data source 106, and the data source 108. It may also manage user accounts, authentication, and the delivery of notifications, such as drug recall notifications or SMS notifications to accountability partners.

The service engine 120 may be configured to manage the operational logic and backend processes of the AI/ML-driven backend system 102. It may act as a central orchestrator, coordinating data flows and interactions between the user-facing application 114, external data sources such as the data source 106 and the data source 108, and the internal components of the AI/ML-driven backend system 102. For example, when the user device 104 transmits first health data, the service engine 120 may be configured to receive this data, validate it, and direct it to the data store 128 for aggregation into the appropriate Consumer Health Record (CHR). The service engine 120 may also manage application programming interface (API) endpoints that facilitate data ingestion from various sources.

In some implementations, the service engine 120 may be built upon a microservices architecture, where individual services handle distinct functionalities. For example, one microservice within the service engine 120 may be configured to manage user authentication and session management, while another may handle the logic for generating and sending notifications. This architectural approach may facilitate scalability and maintainability of the platform. The service engine 120 may be configured to process requests from the secure data exchange system 118, retrieve data as permitted by the consent management system 124, and interact with the AI/ML engine 122 to trigger the generation of personalized care plans or analytics.

Furthermore, the service engine 120 may be configured to execute specific business rules and workflows defined within the platform. For example, it may be configured for tracking medication adherence by processing data received from a pharmacy POS system via the secure data exchange system 118. Based on a medication adherence metric derived from the CHR, the service engine 120 may be configured to transmit an SMS notification to an accountability partner designated by the user. In another example, upon receiving a drug recall alert from an external source, the service engine 120 may query the data store 128 to identify affected users and subsequently transmit a drug recall notification to the appropriate user device 104 or a healthcare provider's system.

The AI/ML engine 122 may be configured to perform advanced analytics on the aggregated CHR data. The AI/ML engine 122 may include at least one of an AI algorithm or a machine learning (ML) algorithm to generate a personalized care plan, predict a potential medication side effect, or generate granular analytics. For example, the AI/ML engine 122 may use a recurrent neural network (RNN) to analyze time-series data from a wearable device to predict a health event.

The AI/ML engine 122 may be architected as a modular system including multiple machine learning models and analytical components, each configured for a specific task. For example, one component may include a predictive model configured for predicting a potential medication side effect. This model may be a supervised learning model, such as a logistic regression, support vector machine, or a gradient boosting machine (e.g., XGBoost), trained on a large dataset of historical, de-identified CHR data. The features for this model may include user demographics, prescribed medications, user-reported health metrics, physiological measurements from wearable devices, and genomic data. The model may be trained to output a probability score indicating the likelihood of a user experiencing a known adverse reaction to a specific medication or combination of medications.

In some implementations, the AI/ML engine 122 may be configured for generating a personalized care plan by utilizing a combination of clustering and recommendation algorithms. Initially, an unsupervised clustering algorithm, such as k-means or hierarchical clustering, may be used to segment users into distinct cohorts based on their comprehensive CHR profiles, including clinical history, lifestyle data from wearable devices, and medication adherence patterns. Once a user is assigned to a specific cohort, a recommendation engine, such as one based on collaborative filtering or a content-based filtering approach, may be configured to suggest specific interventions, educational materials, or lifestyle modifications. For instance, the recommendation engine may analyze successful outcomes from other users within the same cohort to generate data-driven recommendations for a personalized care plan.

Furthermore, the AI/ML engine 122 may be configured for generating granular analytics, such as a health trend insight, by employing time-series analysis models. For analyzing the continual, periodic, or trigger-based data streams from the data source 106, which may include a wearable device, the AI/ML engine 122 may utilize models such as Autoregressive Integrated Moving Average (ARIMA) or an RNN, including Long Short-Term Memory (LSTM) networks. These models may be configured to identify statistically relevant patterns, anomalies, or trends in a user's physiological measurements over time. The output of this analysis may be presented to the user via the UI 116 as a health trend insight, for example, visualizing improvements in resting heart rate correlated with a new medication regimen or flagging a concerning trend in blood glucose levels. The AI/ML engine 122 may be configured to continually retrain its models as new data is aggregated into the data store 128, facilitating adaptive and progressively more accurate analytical outputs.

The consent management system 124 may be configured to manage user permissions for data sharing. The consent management system 124 may provide a dynamic consent management system that facilitates modification, by the user, of data sharing permissions. The AI/ML-driven backend system 102 may determine the governance rules for data exchange by accessing the consent management system 124. For example, a user may use the app 114 to set a governance rule via the consent management system 124 that grants a specific provider read-only access to their CHR for a limited duration.

The consent management system 124 may be architecturally designed as a dedicated service or module within the AI/ML-driven backend system 102, configured to manage and enforce user-defined data sharing preferences, which are stored as governance rules in the data store 128. This system may expose a set of secure APIs that the application 114, via the UI 116, may call to present consent options to the user. For example, when a user accesses the settings portion of the application 114, the consent management system 124 may be configured to retrieve the user's current governance rules from the data store 128 and populate the UI 116 with interactive controls, such as toggles, dropdown menus, and date pickers. These controls may facilitate modification, by the user, of data sharing permissions, including specifying which external healthcare systems (e.g., the healthcare system 110) may access their data, what specific portions of the CHR may be shared (e.g., medication history, physiological measurements, or genomic data), and for what duration the access is granted.

In some implementations, the consent management system 124 may be configured to handle granular, attribute-based access control. For instance, a user may set a governance rule that grants a primary care physician full read-only access to their entire CHR, while granting a specialist access only to data relevant to a specific condition, and simultaneously permitting a research organization to access only de-identified data. When the secure data exchange system 118 receives a data access request from an external entity, such as the healthcare system 110, it may be configured to query the consent management system 124 to validate the request against the established governance rules. The consent management system 124 may then be configured to process the request, verify the identity and permissions of the requesting entity, check for an expiration date for data access, and return an authorization or denial response to the secure data exchange system 118. This interaction ensures that no data is exchanged without explicit, current, and specific user consent.

Furthermore, the consent management system 124 may be configured to manage consent for data monetization and sharing with industry partners. The system may present the user with an option, via the UI 116, to opt-in to a data-sharing agreement with an industry partner in exchange for compensation. The consent management system 124 may be configured to record this consent and apply the corresponding governance rule, which may stipulate that any data shared under this agreement is first processed to be de-identified to protect user privacy. The consent management system 124 may also be configured to generate consent receipts or records for each user action, which may then be passed to the blockchain component 126 to be recorded as an immutable transaction on the audit trail, providing a transparent and verifiable history of all consent modifications.

The blockchain component 126 may be configured to maintain a secure and immutable audit trail of data transactions. The blockchain component 126 may be used for maintaining an audit trail of transactions related to the bi-directional exchange of the at least a portion of the CHR. This creates a transparent and verifiable log of all data access and sharing events, enhancing user trust and data security. For example, every time a portion of the CHR is shared with the healthcare system 110, a transaction may be recorded on a distributed ledger managed by the blockchain component 126.

The blockchain component 126 may be a distributed ledger technology system configured for maintaining, using a blockchain component, an audit trail of transactions related to the bi-directional exchange of the at least a portion of the CHR. This audit trail provides a transparent, verifiable, and immutable record of every data access request, consent modification, and data sharing event that occurs within the AI/ML-driven backend system 102. For each transaction facilitated by the secure data exchange system 118, the blockchain component 126 may be configured to create a new block containing transaction details, such as a timestamp, the identities of the involved parties (e.g., the user and the healthcare system 110), the specific data elements accessed, and a cryptographic hash of the previous block. This chaining of blocks ensures the integrity and chronological order of the audit trail.

In some implementations, the blockchain component 126 may be a private or permissioned blockchain, where access to participate in the network is restricted to authorized entities, such as the AI/ML-driven backend system 102 and potentially trusted third-party auditors. This architectural choice enhances data privacy and control while still leveraging the security benefits of distributed ledger technology. Smart contracts may be deployed on the blockchain to automate the enforcement of governance rules managed by the consent management system 124. For example, a smart contract could be configured to automatically revoke data access for the healthcare system 110 once an expiration date for data access, as specified in a user's governance rule, has passed. This automation reduces the potential for unauthorized data access and ensures that user consent is programmatically enforced.

Furthermore, the blockchain component 126 provides users with a transparent mechanism to verify how their data has been used. Through the application 114, a user may be able to view a simplified representation of the audit trail corresponding to their CHR, showing which entities have accessed their data and when. For example, when a user opts-in to a data-sharing agreement with an industry partner in exchange for compensation, the transaction record, including the de-identification of the data and the terms of the agreement, may be immutably recorded by the blockchain component 126. This functionality provides a high degree of accountability and may be configured to build user trust in the platform's data management practices.

The data store 128 may be a repository for storing all data managed by the AI/ML-driven backend system 102. The data store 128 may be configured to store the CHR, user profiles, governance rules, and analytical models. In some implementations, the data store 128 includes one or more databases, such as a relational database, a NoSQL database, or a distributed file system, configured to handle large volumes of heterogeneous health data securely.

The data store 128 may be architected to support the diverse and high-volume data requirements of the AI/ML-driven backend system 102. In some implementations, the data store 128 may be a multi-modal database system, including a combination of different database technologies to optimize for storage, retrieval, and analysis of various data types. For example, the data store 128 may include a relational database, such as PostgreSQL or MySQL, for storing structured user profile information, authentication credentials, and the governance rules managed by the consent management system 124. For the Consumer Health Record (CHR) itself, which includes heterogeneous data such as time-series physiological measurements, user-reported health metrics, and clinical documents, a NoSQL database may be utilized. Examples of a suitable NoSQL database include a document-oriented database like MongoDB or a wide-column store like Apache Cassandra, which are configured to handle large volumes of unstructured and semi-structured data with high availability and scalability.

In some implementations, the data store 128 may be configured with a data lake architecture to ingest and store raw data from all sources, including the user device 104, the data source 106, and the data source 108, in its native format. This data lake may be built on a distributed file system, such as Hadoop Distributed File System (HDFS) or a cloud-based object storage service like Amazon S3. An extract, transform, load (ETL) pipeline, managed by the service engine 120, may be configured to process this raw data, normalize it into a consistent schema, and load it into a structured data warehouse or the NoSQL database for consumption by the AI/ML engine 122. For example, data received in FHIR or HL7 format from an external health record data source may be parsed, transformed, and aggregated into the user's CHR data structure within the data store 128.

To facilitate security and compliance with healthcare regulations, the data store 128 may be configured with robust security measures. All data at rest within the data store 128 may be encrypted using industry-standard encryption algorithms, such as Advanced Encryption Standard (AES)-256. Access to the data store 128 may be strictly controlled through role-based access control (RBAC) policies, ensuring that components such as the AI/ML engine 122 or the secure data exchange system 118 only have access to the data necessary for their functions. Furthermore, the data store 128 may be partitioned to logically and physically separate identified user data from de-identified data sets used for analytics and industry partner sharing, providing an additional layer of privacy protection. The data store 128 may also be configured for high availability and disaster recovery, using techniques such as database replication and regular backups to protect against data loss.

In operation, the components of the computing environment 100 work in concert to provide a comprehensive and interactive digital health management platform. The process may begin when a user interacts with the application 114 on the user device 104. Through the UI 116, the user may input first health data, such as a prescription indicator or a user-reported health metric. This data is transmitted via the network 112 to the service engine 120 within the AI/ML-driven backend system 102. The data source 106, such as a wearable device, may transmit second health data including at least one physiological measurement to the AI/ML-driven backend system 102. The service engine 120 also initiates requests to external data sources, such as the data source 108 (e.g., an EMR system), to retrieve third health data. The service engine 120 orchestrates the aggregation of the first health data, the second health data, and the third health data, storing the combined information as a unified Consumer Health Record (CHR) in the data store 128.

Once the CHR is populated, the AI/ML engine 122 may be configured to perform advanced analytics. The AI/ML engine 122 may analyze the aggregated CHR data to generate outputs such as a personalized care plan, predict a potential medication side effect, or generate granular analytics including a health trend insight. These analytical results are then passed back to the service engine 120, which may be configured to store them in the data store 128 and present them to the user via the application 114 and the UI 116 on the user device 104. For example, a personalized care plan may be transmitted to a healthcare provider associated with the external healthcare system 110. One or more aspects of the process may be governed by user-defined permissions managed through the consent management system 124. When a data exchange is requested, the secure data exchange system 118 consults the consent management system 124 to determine the applicable governance rules before facilitating any bi-directional exchange with an external entity like the healthcare system 110.

To facilitate transparency and security, every transaction involving the CHR may be digitally recorded by the blockchain component 126. When the secure data exchange system 118 facilitates an exchange of at least a portion of the CHR with the healthcare system 110, it triggers the blockchain component 126 to create an immutable entry in the audit trail. This integration of data aggregation, AI-driven analysis, user-governed data exchange, and blockchain-based auditing facilitates a secure, dynamic, and personalized health management experience. For instance, the system may facilitate remote user monitoring by sharing the second health data with a telehealth system during a virtual consultation, with the consent for this sharing managed by the consent management system 124 and the transaction recorded by the blockchain component 126. In another configuration, the AI/ML engine 122, service engine 120, and data store 128 may be co-located on a single high-performance computing server to minimize latency during data analysis, while the secure data exchange system 118 and consent management system 124 may be deployed as separate, scalable microservices in a cloud environment to handle a high volume of external data requests.

FIG. 2 shows a block diagram of an example of a computing device 200 capable of performing functions described herein. The computing device 200 may be used to implement one or more components of the computing environment 100. The computing device 200 may be, be similar to, include, or be included in an apparatus for performing one or more methods, processes, algorithms, operations, tasks, and/or techniques, as described herein. The computing device 200 may be, be similar to, include, or be included in a computing device, a laptop, a workstation, a server device, or a personal computer, among other examples. The computing device 200 includes a bus 202 that interconnects various components or units, such as a processor 204, a memory 206, a power source 208, an input component 210, an output component 212, and a communication component 214, among other examples.

The processor 204 may be a central processing unit, such as a microprocessor, and may include single or multiple processors having single or multiple processing cores. The processor 204 may include another type of device, or multiple devices, configured for manipulating or processing information. In some implementations, the processor 204 may include one or more processors (which may be referred to herein as a “processor set”) capable of being programmed to perform a function. For example, the processor 204 may include multiple processors interconnected in one or more manners, including hardwired or networked. The operations of the processor 204 may be distributed across multiple devices or units that can be coupled directly or across a local area or other suitable type of network. The processor 204 may include a cache, or cache memory, for local storage of operating data or instructions. The processor 204 may be implemented in hardware, firmware, or a combination of hardware and software.

The processor 204 may include one or more chiplets, chips, system-on-chips (SoCs), network-on-chips (NoCs), chipsets, packages, or devices that individually or collectively constitute or include the processor set. The processor set may include a processor (or “processing”) circuitry in the form of one or multiple processors, microprocessors, processing units (such as CPUs), GPUs, neural processing units (NPUs) and/or digital signal processors (DSPs)), processing blocks, application-specific integrated circuits (ASIC), programmable logic devices (PLDs) (such as field programmable gate arrays (FPGAs)), or other discrete gate or transistor logic or circuitry (all of which may be generally referred to herein individually as “processors” or collectively as “the processor” or “the processor set”).

One or more of the processors of the processor set (e.g., the processor 204) may be individually or collectively configurable or configured to perform various operations described herein. In some implementations, a single processor may perform all of the operations described as being performed by the one or more processors. In some implementations, a group of processors collectively configurable or configured to perform a set of operations may include a first set of (one or more) processors configurable or configured to perform a first operation of the set and a second processor configurable or configured to perform a second operation of the set, or may include the group of processors all being configured or configurable to perform the set of operations. The first set of processors and the second set of processors may be the same set of processors or may be different sets of processors.

The memory 206 includes one or more memory components, which may each be volatile memory or non-volatile memory, that individually or collectively constitute a memory system. The memory system may include memory circuitry in the form of one or more memory devices, memory blocks, memory elements or other discrete gate or transistor logic or circuitry, each of which may include tangible storage media such as random-access memory (RAM) or read-only memory (ROM), or combinations thereof (all of which may be generally referred to herein individually as “memories” or collectively as “the memory,” “the memory system,” or “the memory circuitry”). The memory 206 may include non-transitory memory, transitory memory, or a combination thereof. Volatile memory may include RAM (e.g., a dynamic RAM (DRAM) module, such as a double data rate (DDR) synchronous DRAM (SDRAM)). Non-volatile memory may include a disk drive, a solid state drive, flash memory, or phase-change memory. In some implementations, the memory 206 may be distributed across multiple devices. For example, the memory 206 may include network-based memory or memory in multiple clients or servers performing the operations of those multiple devices.

The memory 206 may be referred to as a computer-readable medium. A computer-readable medium may include any storage unit (or multiple storage units) that store data or instructions that are readable by a processor (e.g., the processor 204). A computer-readable medium may include, for example, at least one of a data repository, a data storage unit, a computer memory, a hard drive, a disk, or a random access memory. As used herein, the term “computer-readable medium” encompasses one or more computer readable media. A computer-readable medium may be a transitory computer-readable medium or a non-transitory computer-readable medium.

One or more of the memories may be coupled (for example, operatively coupled, communicatively coupled, electronically coupled, or electrically coupled) with one or more of the processors and may individually or collectively store processor-executable instructions (e.g., code such as software) that, when executed by one or more of the processors, may configure or otherwise cause one or more of the processors to perform various functions or operations described herein. “Software” shall be construed broadly to mean instructions, instruction sets, code, code segments, program code, programs, subprograms, software modules, applications, software applications, software packages, routines, subroutines, objects, executables, threads of execution, procedures, and/or functions, among other examples, whether referred to as software, firmware, middleware, microcode, hardware description language, or otherwise.

In some implementations, the executable instructions may include application data or an operating system, among other examples. The executable instructions may include one or more application programs, which may be loaded or copied, in whole or in part, from non-volatile memory to volatile memory to be executed by the processor 204. For example, the executable instructions may include instructions for performing techniques described in this disclosure. In some implementations, the application data may include functional programs, such as computational programs, analytical programs, or database programs, among other examples. The operating system may be, for example, Microsoft Windows®, Mac OS X®, or Linux®; an operating system for a mobile device, such as a smartphone or tablet device; or an operating system for a non-mobile device, such as a mainframe computer.

Reference to “one or more memories” should be understood to refer to any one or more memories of a corresponding device, such as the memory described in connection with FIG. 2. For example, operation described as being performed by, or data described as being stored on, one or more memories can be performed by, or stored on, respectively, the same subset of the one or more memories or different subsets of the one or more memories. Additionally or alternatively, in some examples, one or more of the processors may be preconfigured to perform various functions or operations described herein without requiring configuration by software. For example, the memory 206 may include data or instructions that are hard-wired into the processing system.

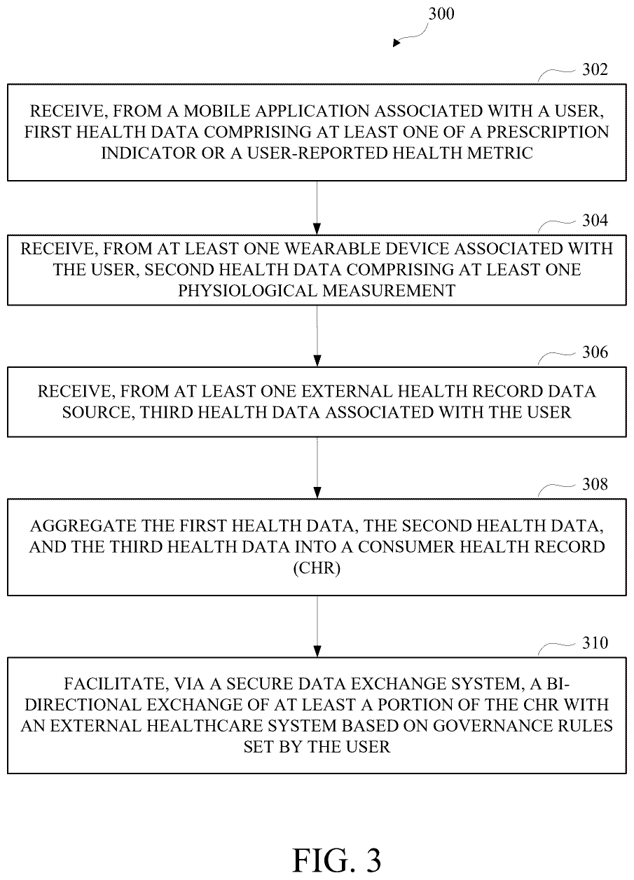

In some implementations, the processor 204 may implement one or more techniques or perform one or more operations associated with providing AI/ML-driven digital health management, as described in more detail elsewhere herein. For example, the processor 204 may perform or direct operations of, for example, technique 300 of FIG. 3 or other techniques as described herein (alone or in conjunction with one or more other processors). In some examples, the memory 206 may include a non-transitory computer-readable medium storing a set of instructions (for example, code or program code). The set of instructions, when executed (for example, directly, or after compiling, converting, or interpreting) by the processor 204, may cause the processor 204 to cause the computing device 200 to perform technique 300 of FIG. 3 or other techniques as described herein. In some examples, executing instructions may include running the instructions, converting the instructions, compiling the instructions, and/or interpreting the instructions, among other examples.

In the description herein, language describing a system, an apparatus, or a device as taking an action (such as performing, determining, initiating, receiving, calculating, deciding, computing, processing, etc.) is to be understood as describing that some appropriate component of the system, apparatus, or device is taking the action. As used herein, the term “component” is intended to be broadly construed as hardware and/or a combination of hardware and software.

An “engine” refers to a component constructed, programmed, configured, or otherwise adapted to perform a specific function or set of functions. The term engine as used herein means a tangible device, component, or arrangement of components implemented using hardware, such as by an ASIC or field-programmable gate array (FPGA), for example, or as a combination of hardware and software, such as by a processor-based computing platform and a set of program instructions that transform the computing platform into a special-purpose device to implement the particular functionality. An engine may also be implemented as a combination of the two, with certain functions facilitated by hardware alone, and other functions facilitated by a combination of hardware and software.

In an example, the software may reside in executable or non-executable form on a tangible machine-readable storage medium. Software residing in non-executable form may be compiled, interpreted, translated, or otherwise converted to an executable form prior to, or during, runtime. In an example, the software, when executed by the underlying hardware of the engine, causes the hardware to perform the specified operations. Accordingly, an engine is physically constructed, or specifically configured (e.g., hardwired), or temporarily configured (e.g., programmed) to operate in a specified manner or to perform part or all of any operations described herein in connection with that engine.

Considering examples in which engines are temporarily configured, each of the engines may be instantiated at different moments in time. For example, where the engines include a general-purpose hardware processor core configured using software, the general-purpose hardware processor core may be configured as respective different engines at different times. Software may accordingly configure a hardware processor core, for example, to constitute a particular engine at one instance of time and to constitute a different engine at a different instance of time.

In certain implementations, at least a portion, and in some cases, all, of an engine may be executed on the processor(s) of one or more computers that execute an operating system, system programs, and application programs, while also implementing the engine using multitasking, multithreading, distributed (e.g., cluster, peer-peer, cloud, etc.) processing where appropriate, or other such techniques. Accordingly, each engine may be realized in a variety of suitable configurations, and should generally not be limited to any particular implementation exemplified herein, unless such limitations are expressly called out. s used herein, the term “model” encompasses its plain and ordinary meaning. A model may include, among other things, one or more engines which receive an input and compute an output based on the input.

The power source 208 provides power to the computing device 200. For example, the power source 208 may be an interface to an external power distribution system. In an example, the power source 208 may be a battery, such as where the computing device 200 is a mobile device or is otherwise configured to operate independently of an external power distribution system. In some implementations, the computing device 200 may include or otherwise use multiple power sources. In some such implementations, the power source 208 can be a backup battery.

The input component 210 and/or the output component 212 may include one or more input interfaces and/or output interfaces configured for facilitating communication between the computing device 200 and one or more peripheral devices such as, for example, one or more sensors, detectors, displays, input devices, or other devices configured for facilitating interaction with the computing device 200 or the environment around the computing device 200. An input device may, for example, include a positional input device, such as a mouse, touchpad, touchscreen, or the like; a keyboard; or another suitable human or machine interface device. An output device may, for example, include a display, such as a liquid crystal display, a cathode-ray tube, a light emitting diode display, or other suitable display. In some implementations, the peripherals devices may include a geolocation component, such as a global positioning system (GPS) location unit. In some examples, the peripheral devices may include a temperature sensor for measuring temperatures of components of the computing device 200, such as the processor 204.

The communication component 214 may include an interface for facilitating a connection or link to a network. The communication component 214 may include a wired network interface or a wireless network interface. The computing device 200 may communicate with other devices via the communication component 214 using one or more network protocols, such as using Ethernet, TCP, IP, power line communication, an IEEE 802.X protocol (e.g., Wi-Fi, Bluetooth, or ZigBee), infrared, visible light, general packet radio service (GPRS), global system for mobile communications (GSM), code-division multiple access (CDMA), Z-Wave, a cellular communication protocol, another protocol, or a combination thereof. For example, the computing device 200 can communicate with a database server.

The communication component 214 may include a transceiver, which may include a transmitter or a receiver. In some configurations, one or a combination of antenna(s), modem(s), multiple input multiple output (MIMO) detectors, receive processors, transmit processors, and/or the transmit MIMO processors may be included in the transceiver. The transceiver may be under control of or used by one or more processors, and in some aspects in conjunction with processor-readable code stored in the memory, to perform aspects of the methods, processes, techniques, and/or operations described herein.

The number and arrangement of components shown in FIG. 2 are provided as an example. The computing device 200 may include additional components, fewer components, different components, or differently arranged components than those shown in FIG. 2. Additionally, or alternatively, a set of components (e.g., one or more components) of the computing device 200 may perform one or more functions described as being performed by another set of components of the computing device 200.

FIG. 3 is a flow diagram illustrating an example of a technique associated with providing AI/ML-driven digital health management. The technique 300 may be executed using computing devices, such as the systems, hardware, and software described with respect to FIGS. 1 and 2. The technique 300 may be performed, for example, by executing a machine-readable program or other computer-executable instructions, such as routines, instructions, programs, or other code. The steps, or operations, of the technique 300, or another technique, method, process, or algorithm described in connection with the implementations disclosed herein may be implemented directly in hardware, firmware, software executed by hardware, circuitry, or a combination thereof.

For simplicity of explanation, the technique 300 is depicted and described herein as a series of steps or operations. However, the steps or operations of the technique 300 may occur in various orders and/or concurrently. Additionally, other steps or operations not presented and described herein may be used. Furthermore, not all illustrated steps or operations may be required to implement a technique in accordance with the disclosed subject matter. The technique 300 may be performed by one or more components of the computing environment 100, such as by the AI/ML-driven backend system 102.

At 302, the technique 300 includes receiving, from a mobile application associated with a user, first health data comprising at least one of a prescription indicator or a user-reported health metric. For example, the service engine 120 of the AI/ML-driven backend system 102 may be configured to receive the first health data from the application 114 executing on the user device 104 via the network 112. The prescription indicator may be generated when a user scans a prescription label using the application 114, and the user-reported health metric may be entered by the user via the UI 116.

At 304, the technique 300 includes receiving, from at least one wearable device associated with the user, second health data comprising at least one physiological measurement. For example, the service engine 120 may be configured to receive the second health data from the data source 106, which may be a wearable device such as a smartwatch or a continuous glucose monitor. This data may be transmitted from the data source 106 to the AI/ML-driven backend system 102, either directly or relayed through the user device 104.

At 306, the technique 300 includes receiving, from at least one external health record data source, third health data associated with the user. For example, the service engine 120, via the secure data exchange system 118, may be configured to receive the third health data from the data source 108. The at least one external health record data source may include at least one of an EMR system or an EHR system. The reception of this data may be facilitated by standardized protocols like FHIR or HL7.

At 308, the technique 300 includes aggregating the first health data, the second health data, and the third health data into a Consumer Health Record (CHR). For example, the service engine 120 may be configured to process and combine the received data from operations 302, 304, and 306 into a unified CHR, which is then stored in the data store 128. In some implementations, the technique 300 may include receiving fourth health data including consumer genomics data and aggregating the fourth health data into the CHR.

At 310, the technique 300 includes facilitating, via a secure data exchange system, a bi-directional exchange of at least a portion of the CHR with an external healthcare system based on governance rules set by the user. For example, the secure data exchange system 118 may be configured to manage this bi-directional exchange with the healthcare system 110. Facilitating the bi-directional exchange may be initiated via at least one of a QR code or a web portal. In some implementations, facilitating the bi-directional exchange may include providing a read-only view of the CHR to a healthcare provider associated with the external healthcare system.

In some implementations, the technique 300 may include determining the governance rules based on accessing a dynamic consent management system that facilitates modification, by the user, of data sharing permissions. The consent management system 124 may manage these permissions. The governance rules may include an expiration date for data access. In some implementations, the technique 300 may include providing, via a governance rule setting, an option for the user to opt-in to a data-sharing agreement with an industry partner in exchange for compensation, wherein data shared with the industry partner is de-identified.

The technique 300 may also include generating a personalized care plan based on analyzing the CHR using at least one of an AI algorithm or an ML algorithm. For example, the AI/ML engine 122 may be configured to perform this analysis. Analyzing the CHR may include predicting a potential medication side effect. Facilitating the bi-directional exchange may include transmitting the personalized care plan to at least one of a healthcare provider or a pharmacist. In some implementations, based on consumer genomics data, the technique may include providing a personalized medicine insight that includes a drug interaction prediction.

In some implementations where the external healthcare system includes a pharmacy POS system, the technique 300 may include tracking medication adherence based on data exchanged with the pharmacy POS system. The technique may further include transmitting an SMS notification to an accountability partner designated by the user based on a medication adherence metric derived from the CHR.

Additional operations may be performed as part of technique 300. For example, the technique may include generating granular analytics based on the CHR, where the granular analytics include at least one of a health trend insight, a goal setting monitor, or a data-driven engagement tool. The technique 300 may also include maintaining, using a blockchain component, an audit trail of transactions related to the bi-directional exchange of the at least a portion of the CHR, as may be performed by the blockchain component 126. Furthermore, the technique 300 may include transmitting a drug recall notification to at least one of the user or a prescribing physician based on the CHR.

Some implementations include a method, comprising: receiving, from a mobile application associated with a user, first health data comprising at least one of a prescription indicator or a user-reported health metric; receiving, from at least one wearable device associated with the user, second health data comprising at least one physiological measurement; receiving, from at least one external health record data source, third health data associated with the user; aggregating the first health data, the second health data, and the third health data into a Consumer Health Record (CHR); and facilitating, via a secure data exchange system, a bi-directional exchange of at least a portion of the CHR with an external healthcare system based on governance rules set by the user.

In some implementations, facilitating the bi-directional exchange comprises providing a read-only view of the CHR to a healthcare provider associated with the external healthcare system. In some implementations, the at least one external health record data source comprises at least one of an EMR system or an EHR system. In some implementations, the external healthcare system comprises a pharmacy POS system, and the method comprises tracking medication adherence based on data exchanged with the pharmacy POS system.

In some implementations, the method comprises generating a personalized care plan based on analyzing the CHR using at least one of an AI or an ML algorithm. In some implementations, analyzing the CHR comprises predicting a potential medication side effect. In some implementations, facilitating the bi-directional exchange comprises transmitting the personalized care plan to at least one of a healthcare provider or a pharmacist. In some implementations, facilitating the bi-directional exchange is initiated via at least one of a QR code or a web portal.

In some implementations, the method comprises determining the governance rules based on accessing a dynamic consent management system that facilitates modification, by the user, of data sharing permissions. In some implementations, the governance rules comprise an expiration date for data access. In some implementations, the method comprises generating granular analytics based on the CHR, the granular analytics comprising at least one of a health trend insight, a goal setting monitor, or a data-driven engagement tool. In some implementations, the method comprises maintaining, using a blockchain component, an audit trail of transactions related to the bi-directional exchange of the at least a portion of the CHR. In some implementations, the method comprises providing, via a governance rule setting, an option for the user to opt-in to a data-sharing agreement with an industry partner in exchange for compensation, wherein data shared with the industry partner is de-identified.

Some implementations include a system, comprising: a memory having instructions stored thereon; and a processor configured to execute the instructions to cause the system to: receive, from a mobile application associated with a user, first health data comprising at least one of a prescription indicator or a user-reported health metric; receive, from at least one wearable device associated with the user, second health data comprising at least one physiological measurement; receive, from at least one external health record data source, third health data associated with the user; aggregate the first health data, the second health data, and the third health data into a Consumer Health Record (CHR); and facilitate, via a secure data exchange system, a bi-directional exchange of at least a portion of the CHR with an external healthcare system based on governance rules set by the user.

In some implementations, the processor is configured to execute the instructions to cause the system to facilitate medication distribution based on a crowdsourcing model. In some implementations, to facilitate the bi-directional exchange, the processor is configured to execute the instructions to cause the system to facilitate remote user monitoring by sharing the second health data with a telehealth system during a virtual consultation.

Some implementations include one or more computer-readable media having computer-executable instructions stored thereon, the computer-executable instructions configured to be executed by a processor to cause the processor to perform operations comprising: receiving, from a mobile application associated with a user, first health data comprising at least one of a prescription indicator or a user-reported health metric; receiving, from at least one wearable device associated with the user, second health data comprising at least one physiological measurement; receiving, from at least one external health record data source, third health data associated with the user; aggregating the first health data, the second health data, and the third health data into a Consumer Health Record (CHR); and facilitating, via a secure data exchange system, a bi-directional exchange of at least a portion of the CHR with an external healthcare system based on governance rules set by the user.

In some implementations, the operations comprise: receiving fourth health data comprising consumer genomics data; aggregating the fourth health data into the CHR; and providing a personalized medicine insight based on the consumer genomics data, the personalized medicine insight comprising a drug interaction prediction. In some implementations, the operations comprise transmitting a drug recall notification to at least one of the user or a prescribing physician based on the CHR. In some implementations, the operations comprise transmitting an SMS notification to an accountability partner designated by the user based on a medication adherence metric derived from the CHR.

Even though particular combinations of features are recited in the claims and/or disclosed in the specification, these combinations are not intended to limit the disclosure of various aspects. In fact, many of these features may be combined in ways not specifically recited in the claims and/or disclosed in the specification. Although each dependent claim listed below may directly depend on only one claim, the disclosure of various aspects includes each dependent claim in combination with every other claim in the claim set. As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiples of the same element (e.g., a-a, a-a-a, a-a-b, a-a-c, a-b-b, a-c-c, b-b, b-b-b, b-b-c, c-c, and c-c-c or any other ordering of a, b, and c).

No element, act, or instruction used herein should be construed as critical or essential unless explicitly described as such. Also, as used herein, the articles “a” and “an” are intended to include one or more items and may be used interchangeably with “one or more.” Further, as used herein, the article “the” is intended to include one or more items referenced in connection with the article “the” and may be used interchangeably with “the one or more.” Furthermore, as used herein, the terms “set” and “group” are intended to include one or more items (e.g., related items, unrelated items, or a combination of related and unrelated items), and may be used interchangeably with “one or more.” Where only one item is intended, the phrase “only one” or similar language is used. Also, as used herein, the terms “has,” “have,” “having,” or the like are intended to be open-ended terms. Further, the phrase “based on” is intended to mean “based, at least in part, on” unless explicitly stated otherwise. Also, as used herein, the term “or” is intended to be inclusive when used in a series and may be used interchangeably with “and/or,” unless explicitly stated otherwise (e.g., if used in combination with “either” or “only one of”).

The adjectives “first,” “second,” “third,” and so on are used for contextual distinction between two or more of the modified nouns in connection with a discussion and are not meant to be absolute modifiers that apply only to a certain respective node throughout the entire document. For example, a component may be referred to as a “first component” in connection with one discussion and may be referred to as a “second component” in connection with another discussion, or vice versa. Reference to a component, a computing device, a server, a client, an application, an apparatus, a device, a system, a computing system, or the like may include disclosure of the computing device, server, client, application, apparatus, device, system, computing system, or the like, respectively, being a node. For example, disclosure that a computing device is configured to receive information from a server also discloses that a first node is configured to receive information from a second node. Consistent with this disclosure, once a specific example is broadened in accordance with this disclosure (e.g., a computing device is configured to receive information from a server also discloses that a first node is configured to receive information from a second node), the broader example of the narrower example may be interpreted in the reverse, but in a broad open-ended way. In the example above where a computing device being configured to receive information from a server also discloses a first node being configured to receive information from a second node, “first node” may refer to a first computing device, a first server, a first client, a first application, a first apparatus, a first device, a first system, a first computing system, or the like, configured to receive the information from a second node; and “second node” may refer to a second computing device, a second server, a second client, a second application, a second apparatus, a second device, a second system, a second computing system, or the like.

As used herein, unless explicitly stated otherwise, any term specified in the singular may include its plural version. For example, “a computer that stores data and runs software,” may include a single computer that stores data and runs software or two computers—a first computer that stores data and a second computer that runs software. Also “a computer that stores data and runs software,” may include multiple computers that together stored data and run software. At least one of the multiple computers stores data, and at least one of the multiple computers runs software.

The implementations of this disclosure can be described in terms of functional block components and various processing operations. Such functional block components can be realized by a number of hardware or software components that perform the specified functions. For example, the disclosed implementations can employ various integrated circuit components (e.g., memory elements, processing elements, logic elements, look-up tables, and the like), which can carry out a variety of functions under the control of one or more microprocessors or other control devices. Similarly, where the elements of the disclosed implementations are implemented using software programming or software elements, the systems and techniques can be implemented with a programming or scripting language, such as C, C++, Java, JavaScript, assembler, or the like, with the various algorithms being implemented with a combination of data structures, objects, processes, routines, or other programming elements.

Functional aspects can be implemented in algorithms that execute on one or more processors. Furthermore, the implementations of the systems and techniques disclosed herein could employ a number of conventional techniques for electronics configuration, signal processing or control, data processing, and the like. The words “mechanism” and “component” are used broadly and are not limited to mechanical or physical implementations, but can include software routines in conjunction with processors, etc. Likewise, the terms “system” or “tool” as used herein and in the figures, but in any event based on their context, may be understood as corresponding to a functional unit implemented using software, hardware (e.g., an integrated circuit, such as an ASIC), or a combination of software and hardware. In certain contexts, such systems or mechanisms may be understood to be a processor-implemented software system or processor-implemented software mechanism that is part of or callable by an executable program, which may itself be wholly or partly composed of such linked systems or mechanisms.

While the disclosure has been described in connection with certain implementations, it is to be understood that the disclosure is not to be limited to the disclosed implementations but, on the contrary, is intended to cover various modifications and equivalent arrangements included within the scope of the appended claims, which scope is to be accorded the broadest interpretation so as to encompass all such modifications and equivalent structures as is permitted under the law.

What is claimed is:

1. A method, comprising:
receiving, from a mobile application associated with a user, first health data comprising at least one of a prescription indicator or a user-reported health metric;
receiving, from at least one wearable device associated with the user, second health data comprising at least one physiological measurement;
receiving, from at least one external health record data source, third health data associated with the user;
aggregating the first health data, the second health data, and the third health data into a Consumer Health Record (CHR); and
facilitating, via a secure data exchange system, a bi-directional exchange of at least a portion of the CHR with an external healthcare system based on governance rules set by the user.

2. The method of claim 1, wherein facilitating the bi-directional exchange comprises providing a read-only view of the CHR to a healthcare provider associated with the external healthcare system.

3. The method of claim 1, wherein the at least one external health record data source comprises at least one of an electronic medical record (EMR) system or an electronic health record (EHR) system.

4. The method of claim 1, wherein the external healthcare system comprises a pharmacy Point-of-Sale (POS) system, the method comprising:
tracking medication adherence based on data exchanged with the pharmacy POS system.

5. The method of claim 1, comprising:
generating a personalized care plan based on analyzing the CHR using at least one of an artificial intelligence (AI) or a machine learning algorithm.

6. The method of claim 5, wherein analyzing the CHR comprises:
predicting a potential medication side effect.

7. The method of claim 5, wherein facilitating the bi-directional exchange comprises:
transmitting the personalized care plan to at least one of a healthcare provider or a pharmacist.

8. The method of claim 1, wherein facilitating the bi-directional exchange is initiated via at least one of a quick response (QR) code or a web portal.

9. The method of claim 1, comprising:
determining the governance rules based on accessing a dynamic consent management system that facilitates modification, by the user, of data sharing permissions.

10. The method of claim 9, wherein the governance rules comprise an expiration date for data access.

11. The method of claim 1, comprising:
generating granular analytics based on the CHR, the granular analytics comprising at least one of a health trend insight, a goal setting monitor, or a data-driven engagement tool.

12. The method of claim 1, comprising:
maintaining, using a blockchain component, an audit trail of transactions related to the bi-directional exchange of the at least a portion of the CHR.

13. The method of claim 1, comprising:
providing, via a governance rule setting, an option for the user to opt-in to a data-sharing agreement with an industry partner in exchange for compensation, wherein data shared with the industry partner is de-identified.

14. A system, comprising:
a memory having instructions stored thereon; and
a processor configured to execute the instructions to cause the system to:
receive, from a mobile application associated with a user, first health data comprising at least one of a prescription indicator or a user-reported health metric;
receive, from at least one wearable device associated with the user, second health data comprising at least one physiological measurement;
receive, from at least one external health record data source, third health data associated with the user;
aggregate the first health data, the second health data, and the third health data into a Consumer Health Record (CHR); and
facilitate, via a secure data exchange system, a bi-directional exchange of at least a portion of the CHR with an external healthcare system based on governance rules set by the user.

15. The system of claim 14, wherein the processor is configured to execute the instructions to cause the system to:
facilitate medication distribution based on a crowdsourcing model.

16. The system of claim 14, wherein, to facilitate the bi-directional exchange, the processor is configured to execute the instructions to cause the system to:
facilitate remote user monitoring by sharing the second health data with a telehealth system during a virtual consultation.

17. One or more computer-readable media having computer-executable instructions stored thereon, the computer-executable instructions configured to be executed by a processor to cause the processor to perform operations comprising:
receiving, from a mobile application associated with a user, first health data comprising at least one of a prescription indicator or a user-reported health metric;
receiving, from at least one wearable device associated with the user, second health data comprising at least one physiological measurement;
receiving, from at least one external health record data source, third health data associated with the user;
aggregating the first health data, the second health data, and the third health data into a Consumer Health Record (CHR); and
facilitating, via a secure data exchange system, a bi-directional exchange of at least a portion of the CHR with an external healthcare system based on governance rules set by the user.

18. The one or more computer-readable media of claim 17, the operations comprising:
receiving fourth health data comprising consumer genomics data;
aggregating the fourth health data into the CHR; and
providing a personalized medicine insight based on the consumer genomics data, the personalized medicine insight comprising a drug interaction prediction.

19. The one or more computer-readable media of claim 17, the operations comprising:
transmitting a drug recall notification to at least one of the user or a prescribing physician based on the CHR.

20. The one or more computer-readable media of claim 17, the operations comprising:
transmitting a short message service (SMS) notification to an accountability partner designated by the user based on a medication adherence metric derived from the CHR.
